(19) United States
(12) Patent Application Publication
(10) Pub. No.: US 2004/0176823 A1
(43) Pub. Date: Sep. 9, 2004

(54) Acne treatment device and method

(76) Inventors: Tobin C. Island, Oakland, CA (US); Robert E. Grove, Pleasanton, CA (US); Mark V. Weckwerth, Pleasanton, CA (US)

Correspondence Address: GRAY CARY WARE & FREIDENRICH LLP, 153 TOWNSEND, SUITE 800, SAN FRANCISCO, CA, 94107, US

(21) Appl. No.: 10/788,167

(22) Filed: Feb. 25, 2004

Related U.S. Application Data
(60) Provisional application No. 60/451,091, filed on Feb. 28, 2003.
(60) Provisional application No. 60/456,379, filed on Mar. 20, 2003.
(60) Provisional application No. 60/458,861, filed on Mar. 27, 2003.
(60) Provisional application No. 60/472,056, filed on May 20, 2003.
(60) Provisional application No. 60/450,243, filed on Feb. 25, 2003.
(60) Provisional application No. 60/450,598, filed on Feb. 26, 2003.
(60) Provisional application No. 60/452,304, filed on Mar. 4, 2003.
(60) Provisional application No. 60/451,981, filed on Mar. 4, 2003.
(60) Provisional application No. 60/452,591, filed on Mar. 6, 2003.
(60) Provisional application No. 60/456,586, filed on Mar. 21, 2003.

(51) Int. Cl. A61N 5/06

(52) U.S. Cl. 607/88

(57) ABSTRACT

Disclosed are a method and a dermatological device for treatment of acne that includes an intense violet-blue diode light source and an output window that contacts the skin during the light emission to provide a heat sink for the skin. Another disclosed device configuration and method includes a handheld and cordless device with an intense violet-blue light source and a contact-based heat sink for the skin. A further configuration and method provides a small area illumination and a contact-based heat sink. A still further configuration and method provides a handheld and cordless device having a small area illumination and contact-based heat sink.

### PRIORITY

This application claims the benefit of priority under 35 U.S.C. § 119(e) to U.S. provisional patent applications nos. 60/451,091, filed Feb. 28, 2003; 60/456,379, filed Mar. 20, 2003; 60/458,861, filed Mar. 27, 2003; 60/472,056, filed May 20, 2003; 60/450,243, filed Feb. 25, 2003; 60/450,598, filed Feb. 26, 2003; 60/452,304, filed Mar. 4, 2003; 60/451,981, filed Mar. 4, 2003; 60/452,591, filed Mar. 6, 2003; and 60/456,586, filed Mar. 21, 2003.

### FIELD OF THE INVENTION

The present invention relates to devices and methods for the treatment of acne, and more particularly, to improved light-based devices and methods.

### BACKGROUND OF THE INVENTION

Acne vulgaris and related conditions (hereafter referred to as “acne”) are exceedingly common skin disorders that can cause severe emotional effects and permanent disfigurement. Approximately 85% of individuals between the ages of 12 and 24 suffer from acne, and although the condition generally resolves at the end of adolescence, it persists into adulthood for more than 25% of the population. The severity of the affliction varies widely from individual-to-individual and from time-to-time, ranging from week-long outbreaks of a few non-inflammatory comedos to years of persistent comedos and inflamed cysts that heal by scarring.

Acne is generally thought to be caused by the obstruction of sebaceous follicles by a mixture of excess sebum and desquamated epithelial cells from the follicle walls. The obstruction forms a microcomedo that evolves either into a comedo (commonly known as a blackhead or whitehead) or into an inflammatory lesion (papules, pustules, and cysts). Propionibacterium acnes (P. acnes) or other naturally present organisms can proliferate in the mixture of sebum and epithelial cells and promote inflammation.

The most common current therapy for acne is over-the-counter (OTC) medications and prescription drugs, both of which generally target one or more of the pathogenesis factors: reduction of sebum production, reduction of epithelial desquamation in sebaceous follicles, or reduction of proliferation of P. acnes. The OTC medications include simple cleansers and low concentration topicals, such as salicylic acid to reduce desquamation and benzoyl peroxide for its antibacterial action. Such therapies generally offer minimal to moderate efficacy with relatively low side effects. Prescription medications include systemic estrogens, anti-androgens, and isotretinoin to reduce sebum production; isotretinoin, topical tretinoin, and antibiotics to reduce desquamation; and systemic and topical antibiotics, such as tetracycline, to reduce P. acnes proliferation. They generally provide better efficacy than OTC options, but can have significant limitations and side effects. For example, estrogens and anti-androgens are suitable only for women, anti-androgens can influence fetus development, and oral isotretinion, while highly effective, has been associated with arthralgia, tendonitis, depression, and birth defects. There is also an increasing concern in the medical community that antibiotics are over-prescribed and that P. acnes is becoming resistant to antibiotics.

An appealing alternative or adjunct to drug therapy is the use of light to treat acne. In acne phototherapy, electromagnetic radiation is used to treat the cause and/or symptoms of acne. Various techniques and devices are known and include UV, visible, and infra-red wavelengths; pulsed and continuous wave radiation; and mechanisms of actions that include bio-stimulation, anti-bacterial, and anti-sebaceous. The present invention is related to the use of violet-blue light (400-450 nm) to treat acne. Violet-blue light is believed to be absorbed by endogenous porphyrins produced by the bacteria present in acne lesions, reducing or reversing the proliferation of the bacteria, and thereby helping to clear the lesions.

### CURRENT STATE OF THE ART

Scientific Art

It is scientifically well-established that bacteria are present in acne lesions and produce various porphyrins, including copro- and proto-porphyrin produced by P. acnes. (Cornelius & Ludwig 1967, Lee et al. 1978, Fanta et al. 1981, Melo and Johnsson 1982, Kjeldstad et al. 1984). Porphyrins are well-known ring molecules that are widely prevalent in biological processes, have strong absorption around 400 nm in the Soret band with features that vary slightly with specific porphyrin species (Leung, 1996), and are photosensitizing agents which can induce cell damage after irradiation (Girotti, 1983).

Kjeldstad et al. (1985) studied in vitro the photosensitization of P. acnes due to the endogenous porphyrins and found that P. acnes was inactivated with 415 nm light in proportion to the concentration of porphyrin and suggests the possible clinical treatment of acne with light. In further work, Kjeldstad and Johnsson (1986) report an action spectrum for blue and near-UV photoinactivation of P. acnes. The action spectrum shows a secondary peak near 415 nm, which they attribute to porphyrin absorption, citing the correlation with the peak of the porphyrin absorption and the dependence on porphyrin concentration. The intensity at 415 nm was 5 mW/cm², which they report as about 5 times the intensity of sunlight in the band 410-420 nm. The destruction mechanism for bacteria due to photosensitization of porphyrin may involve the production of singlet oxygen (Ito 1978, Kelland et al 1983, Kjeldstad et al. 1986, Kjeldstad 1984, Arakane et al. 1996). It is also possible that photo-excited porphyrin is itself toxic to bacteria or produces a toxic precursor other than singlet oxygen (R. Rox Anderson, private communication, 2004).

In clinical studies, Meffert et al. (1990) found that repeated irradiations with short range visible light (400-420 nm) with 10 serial irradiations of 10 minutes each at 54 mW/cm² for a total dose of 325 J/cm² improved acne and seborrhea markedly. The Meffert device used a high pressure lamp.

Sigurdsson et al. (1996) conducted clinical studies with 20 serial irradiations from three filtered arc lamps, attempting to separate the effects of UVA, violet-blue, and green light. Treatments were 20 minutes for each of the three sources and the power level for the violet-blue (400-440 nm) case was about 16 mW/cm². All light sources caused improvement with moderate efficacy variation between sources. The violet-blue light appeared better in the researchers' and patients' opinions. They also report that the porphyrin levels in the acne lesion were reduced after violet-blue irradiation, suggesting a reduction in the P. acnes levels.

Several studies have been reported using a metal halide lamp with violet-blue output in the 407-420 nm band and longer visible wavelengths. Shalita et al (2001) demonstrated 60% clearance of acne lesions, following a total of eight bi-weekly 10-minute treatment sessions. They report an output of 90 mW/cm² of visible light and apparently about 20 mW/cm² of 407-420 m violet-blue. Ashkenazi et al. (2002) performed in vitro 407-420 nm irradiation of P. acnes and report photoinactivation of five orders of magnitude with three irradiations of 75 J/cm². The addition of δ-aminolevulinic acid (ALA) to the cultures increased porphyrin concentrations and improved the killing efficiency of the light. X-ray microanalysis and transmission electron microscopy showed structural damage to the membranes of the illuminated P. acnes. Shnitkind et al. found that narrow-band 407-420 nm light has an anti-inflammatory effect on keratinocytes in addition to an anti-microbial effect on P. acnes. Kawada et al. (2002) found that twice weekly treatments of 5 weeks reduced acne lesions by 64%. An in vitro investigation showed the 407-420 nm light was effective at killing P. acnes but not Staphylococcus epidermidis that were isolated from some acne patients.

Papageorgiou et al. (2000) compared clinical results of violet-blue light (peak at 415 nm) alone and violet-blue light combined with red light (peak at 660 nm). Subjects were treated daily for 15 minutes for 12 weeks with an intensity of about 4 mW/cm² for violet-blue light and 3 mW/cm² for red light. The irradiation sources were fluorescent lamps in reflector fixtures. Both sources showed statistically significant reductions in inflammatory lesions and comedone counts compared to the white light control, with an efficacy comparable to 5% benzoyl peroxide. The combined violet-blue and red light resulted in better clearing for inflammatory lesions, which the authors suggest may be due to the anti-inflammatory action of red light.

Patent Art

There are a number of relevant devices and methods for phototherapy disclosed in the patent literature.

Diamantopoulos et al. (U.S. Pat. No. 4,930,504, issued June 1990) describe a device for biostimulation of tissue including an array of substantially monochromatic light sources with a plurality of wavelengths. The light sources are arranged within the array such that at least two wavelengths pass through a single point within the treatment target tissue.

In U.S. Pat. No. 5,259,380 (issued November 1993), Mendes et al. describe a device and method for light therapy that include light emitting diodes (LED's) emitting in the red and infra-red bands and directing the light onto a dermal region. U.S. Pat. No. 5,549,660 (issued August 1996), also to Mendes et al., provides an acne treatment method using a plurality of red band LED's.

Kohler (U.S. Pat. No. 6,183,500, issued February 2001) discloses a method and apparatus for cosmetic treatment of acne utilizing light characterized by a combination of two emission spectra, one in a blue region and the other in a red region.

In PCT application WO 00/02491, published January 2000) and U.S. published application nos. 2001/0023363 (Harth et al.), 2002/0173833 (Korman et al.), 2002/0128695 (Harth et al.), and 2003/0216795, devices and methods are disclosed for acne treatment that include a light source in at least a spectral band of 405-440 nm. Additional wavelength bands and topical solutions are also described.

Wilkens et al. (U.S. published application no. 2002/0161418) describe a light irradiation device for various skin conditions that comprises at least one spectral band between 400-500 nm and comprises certain ranges of power and energy.

In U.S. Pat. No. 5,486,172 (issued January 1996) to Chess, a device is provided for treating cutaneous vascular lesions that includes a means of cooling the skin with a window for the light that is in contact with the skin. U.S. Pat. No. 5,057,104 (issued October 1991) and U.S. Pat. No. 5,282,797 (issued February 1994), also to Chess, also discuss contact cooling for vascular lesion treatment.

Anderson et al. (U.S. Pat. No. 5,735,844, issued April 1998, and U.S. Pat. No. 5,595,568, issued January 1997) describe devices and methods for hair removal that include contact cooling of the skin. U.S. Pat. No. 6,659,999 (issued December, 2003), also to Anderson, describes methods for treating skin wrinkles that include electromagnetic radiation and contemporaneous cooling of the epidermis.

Commercial Art

There are some known commercial devices that are marketed to the medical community and/or consumer that are relevant to the present invention.

One device is the ClearLight (CureLight, Ltd., Margate, Fla.), which employs metal halide lamps as light sources with output in a 405-420 nm band and a dual head treatment area with two 30 cm by 30 cm treatment regions. The device includes a fan that can be directed on the patient's skin to provide cooling. The stated treatment protocol is 15 minute treatments. The ClearLight is FDA-cleared for acne treatment. CureLight also markets a similar, single-head device called the iClear.

Another device is the Omnilux Blue (Photo Therapeutics, Ltd., Cheshire, UK), which has a treatment head with a matrix of LED's providing a 407 nm output at an intensity of 40 mW/cm². The stated treatment protocol is twice weekly sessions for four weeks with a 20 minute exposure to the light. The device provides full face treatment and is FDA-cleared for acne treatment.

There are several devices on the market that have fluorescent tubes that output blue and red light bands, including the DermaLux AV (DermaLux, Ltd., Chatham, UK), the Red'n'Blue (Red'n'Blue—Team Sylvania, Erlangen, Germany), and the Verilux Happy Skin (Verilux, Stamford, Conn.). The devices are marketed for full face treatments with a daily exposure time of 15 minutes.

Dima-Tech (National City, Calif.) markets the Acnelamp, which is a combination blue and red light device with LED-based light source. The device is a table-top lamp with one, two, or three heads on goosenecks that illuminate the face at a distance.

A review of the state of the art shows that violet-blue light can be a safe and effective treatment for acne. However, the existing devices and methods have important deficiencies.

Firstly, the treatment protocol for all of these light-based devices calls for long periods of exposure of the skin to the violet-blue (and any additional wavelength) light emitted by the device. It would be desirable for each of these treatments to be delivered more quickly. However, increasing the intensity of the light output in an effort to reduce the treatment time would result in excessive heating of the skin. Even the fan cooling employed by some of the prior devices is not adequate to maintain the skin within a tolerable temperature range if more intense illumination were used. In addition to lowering treatment times, a more intense output could increase efficacy by providing a higher dose in the same treatment time as a less intense light. Clearly, a device or method that enables the use of a more intense illumination by ensuring that the skin does not overheat is desirable.

Another deficiency with current violet-blue devices and methods is the large area of illumination, namely the entire face, upper back, or shoulders of the patient being treated. Because the presence of the porphyrins produced the P. acnes bacteria is substantially localized to lesions infected by P. acnes there is believed to be little or no benefit to treating unaffected regions. It is undesirable to treat the unaffected skin regions for at least three reasons. Firstly, light with a wavelength at or near 405 nm may contribute to photoaging of the skin. Secondly, a device or method that illuminates large regions must have a more powerful illumination source than a device that illuminates only affected areas of the skin, increasing cost and/or size. Thirdly, it is more difficult to prevent the skin from overheating when a large area is illuminated.

Finally, the currently available devices and methods are inconvenient to use because the devices are large and cumbersome and/or require a power cord to be attached to the device. The size and/or cord limits the ability of the operator to position the devices into orientations that are required to best treat a desired region of skin. Additionally, the large size makes the device difficult to relocate for use in multiple locations, or to be shared among different sites.

### SUMMARY OF THE INVENTION

Our invention improves upon at least one or more of the above deficiencies in the existing state of the art in acne phototherapy. In one embodiment, we disclose a method and device that includes an intense violet-blue diode light source and an output window that contacts the skin during the light emission to provide a heat sink for the skin. In another embodiment, we disclose a handheld and cordless device with an intense violet-blue light source and a contact-based heat sink for the skin. In a third embodiment, we disclose a method and device with small area illumination and contact-based heat sink. A fourth embodiment provides a handheld and cordless device having a small area illumination and contact-based heat sink.

It is therefore an object of the present invention to provide a method and device for treatment of acne or other skin condition and which employs intense violet-blue light and an output window that operates as a heat sink upon contact with skin undergoing treatment.

It is another object of the present invention to provide a method and handheld, cordless device which is capable of emitting intense violet-blue light and which provides contact-based heat removal for skin undergoing treatment.

It is a further object of the present invention to provide a method and devices emitting intense violet-blue light in a small illumination area and an output window or other structure which provides contact based heat sinking.

These and other objectives, advantages and features of the present invention will be more readily understood upon considering the following detailed description of certain preferred embodiments of the present invention, and the accompanying drawings.

### INCORPORATION BY REFERENCE

What follows is a list of citations corresponding to references which are, in addition to those references cited above and below, and including that which is described as background and the invention summary, hereby incorporated by reference into the detailed description of the preferred embodiments below, as disclosing alternative embodiments of elements or features of the preferred embodiments that may not otherwise be set forth in detail below. A single one or a combination of two or more of these references may be consulted to obtain a variation of the elements or features of preferred embodiments described in the detailed description below. Further patent, patent application and non-patent references are cited in the written description and are also incorporated by reference into the preferred embodiment with the same effect as just described with respect to the following references:

U.S. Pat. Nos. 4,930,504; 5,057,104; 5,259,380; 5,282,797; 5,486,172; 5,549,660; 5,595,568; 5,735,844; 6,183,500; 6,659,999;

United States published application nos. 2001/0023363; 2002/0128695; 2002/0161418; 2002/0173833; 2003/0216795;

U.S. provisional patent applications nos. 60/451,091, filed Feb. 28, 2003; 60/456,379, filed Mar. 20, 2003; 60/458,861, filed Mar. 27, 2003; 60/472,056, filed May 20, 2003; 60/450,243, filed Feb. 25, 2003; 60/450,598, filed Feb. 26, 2003; 60/452,304, filed Mar. 4, 2003; 60/451,981, filed Mar. 4, 2003; 60/452,591, filed Mar. 6, 2003; and 60/456,586, filed Mar. 21, 2003, all of which are assigned to the assignee of the subject application (collectively, the “Cross-Referenced Provisional Applications”);

U. S. non-provisional patent application no. ______, filed Feb. ______, 2004, entitled “Self-Contained Eye-Safe Hair-Regrowth-Inhibition Apparatus And Method,” naming as inventors Tobin C. Island, Robert E. Grove, and Mark V. Weckwerth; Ser. No. ______, filed Feb. ______, 2004, entitled “Eye-Safe Dermatologic Treatment Apparatus And Method,” naming as inventors: Robert E. Grove, Mark V. Weckwerth, Tobin C. Island; and Ser. No.______, filed Feb. ______, 2004, entitled “Self-Contained, Diode-Laser-Based Dermatologic Treatment Apparatus And Method,” naming as inventors: Mark V. Weckwerth, Tobin C. Island, Robert E. Grove, all of which are assigned to the assignee of the subject application (collectively “the Cross-Referenced Non-Provisional Applications”);

Published PCT application no. WO 00/02491;

Scientific publications—see the Scientific Publications List located at the end of the Detailed Description of the Preferred Embodiments section, herein.

Attention is drawn to the aforementioned Cross-Referenced Provisional Applications and Cross-Referenced Non-Provisional Applications by the same inventors of the subject application that disclose various aspects of dermatologic devices, including hair removal devices and methods and eye safety devices and methods. It is clear that one of ordinary skill in the art will recognize that aspects and features disclosed in those applications may be configured so as to be suitable for use in the acne treatment device and method described herein.

### BRIEF DESCRIPTION OF THE DRAWINGS

FIG. 1 is a schematic illustration of one embodiment of the invention.

FIG. 2 is a graphical illustration of the results of a skin temperature calculation for a first set of conditions.

FIG. 3 is a graphical illustration of the results of a skin temperature calculation for a second set of conditions.

FIG. 4 is a graphical illustration of the results of a skin temperature calculation for a third set of conditions.

FIG. 5 is a graphical illustration of the results of a skin temperature calculation for a fourth set of conditions.

FIG. 6 is a schematic illustration of one embodiment of a light source comprising light emitting diodes which is suitable for use in the invention.

### DETAILED DESCRIPTION OF THE PREFERRED EMBODIMENTS

Preliminarily, it is to be noted that the terms “blue” and “violet” light generally refer to wavelengths bands in the range of 420-490 nm and 380-420 nm, respectively, although this terminology is not universally applied in the art. Blue, violet, blue-violet, or violet-blue designations can be considered generally equivalent for purposes of the subject application. It is also to be understood that the term “light,” when not otherwise qualified, is used herein to encompass electromagnetic radiation, including radiation in the UV, visible, and infra-red regions, and not merely the visible spectrum.

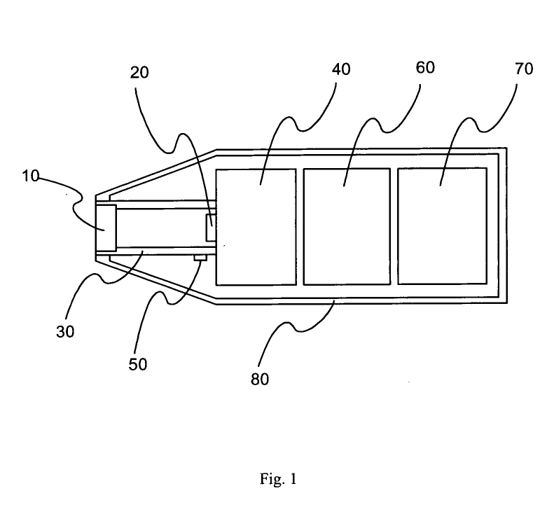

A schematic of a preferred embodiment of the device is shown in FIG. 1. In this embodiment, the device is contained within a housing 80 that includes an output window 10 through which intense violet-blue light can be delivered to a region of the skin. Prior to the light emission, window 10 is placed in intimate contact with the region of skin to be treated. During the emission, window 10 is held in contact with the skin. After emission, the window may be repositioned to a new region of skin and the treatment can be repeated.

One purpose of window 10 is to transmit the light produced by the light source 20 to the region of the skin to be treated. Therefore, window 10 must be formed of a material transparent to the therapeutic wavelengths produced by light source 20. Sapphire is a preferred material but other transparent materials may be used, including fused quartz, fused silica, polymeric materials, opal glass, or glass. By transparent it is meant that the material has a transmissivity at the therapeutic wavelength of at least 50%, although lower transmissivity may be acceptable for various reasons, including the use of diffusive materials such as opal glass to improve uniformity or eye safety or if the light that is not transmitted on the first pass has additional opportunities for transmission, say, because of a reflector surrounding the light source.

Another purpose of window 10 is to provide a heat sink for the skin so that the skin temperature does not increase to a temperature that is high enough to cause excessive discomfort or damage the skin. Violet-blue light is absorbed within a short distance in skin (effective absorption length of approximately 0.3 mm) and causes the skin temperature to increase. Heat transfer from the skin into window 10 mitigates this temperature rise. A 5 mm thick sapphire disk has enough heat capacity and has a high enough thermal diffusivity to accept 25 Joules of heat during a 10 second exposure with a temperature increase of less than 20° C. Materials other than sapphire may be used for window 10; however, a material with high heat capacity yet low thermal diffusivity may not work since the full heat capacity of the material may not be utilized if insufficient thermal diffusion prevents the full volume of the material to be heated within the exposure time of the light.

In a preferred embodiment of the invention, window 10 is at or near the nominal skin temperature prior to contact with the skin and does not substantially cool the surface of the skin below its nominal temperature. The nominal skin temperature is the temperature of the skin prior to contact or illumination, and is generally around 32 to 35 C. In this case, the window does not pre-cool the skin but serves as a heat sink during light emission so as to prevent the skin from reaching too high a temperature. In a preferred embodiment, the heatsink would limit the maximum temperature rise in the epidermis to less than about 25 C.

Another embodiment of the invention involves cooling window 10 to a temperature below the nominal skin temperature, for example to a temperature between 0 C and the nominal skin temperature. When window 10 is placed in contact with the skin prior to light emission, the skin is pre-cooled by the window to lower the skin temperature below the nominal skin temperature. During the light emission, the window 10 provides heat sinking for the skin that is concurrent with the emission.

The most preferred area dimension for window 10 is about 1 cm² so that small regions of skin like the side of the nose or even individual acne lesions can be treated. In another embodiment of the current invention, window 10 may be as large as 5 cm² or even 25 cm² so as to be able to treat a number of lesions or somewhat larger area at a time. However, the maximum size of window 10 is limited by the need for the entire area of the window to be in contact with skin so that it can provide a heat sink to the entire region of skin being illuminated. Too large a window would not conform to the skin where the body is curvaceous, such as regions of skin on or near the nose and upper lip.

The term “spot size” as used in this document refers to the area of the treatment beam at the emitting surface of window 10. The perimeter of this area may be defined by the locations where the intensity of the treatment beam drops to 1/e² of the intensity at the center of the spot. The output window 10 may have a larger size than the spot size in order, for example, to accommodate an optical skin sensor, or may have a different geometry, for example the treatment beam is square and the output window 10 is round for lower cost and ease of manufacturing. In one embodiment, the spot size is about 0.81 cm² with a square cross-section and the window is circular with an area of about 1.3 cm².

One embodiment of the invention includes a mixer 30 that is used to make the light emitted by the light source 20 more spatially uniform upon illuminating the skin. It is desirable for the spatial uniformity of the illumination at the skin to have a variation of less than +/−40% so that all of the treated skin receives a similar dose of light. In a preferred embodiment, mixer 30 is a hollow aluminum tube with square cross-section about 2 cm in length. The walls of mixer 30 are substantially non-absorbing at the therapeutic wavelengths emitted by source 20 so that light impinging upon the walls of mixer 30 is reflected. As the light travels through mixer 30 from light source 20 to output window 10, the spatial uniformity of the light increases. The length, maximum absorption, and cross-sectional geometry of mixer 30 required for sufficient mixing of the light are dependent upon the size of window 10 and the size and output characteristics of light source 20. Additional details and considerations of mixer design can be found in the above referenced Cross-Referenced Non-Provisional Applications.

In another embodiment, mixer 30 could be a solid light guide in which light from source 20 is totally internally reflected along the light guide to window 10. A mixer that is a solid light guide could itself form the exit aperture for the light and thereby serve as window 10.

In another embodiment, it is conceivable that a light source with sufficient uniformity and size could be developed that would make mixer 30 unnecessary.

In a preferred embodiment a two-dimensional array of LED's would be used for light source 20. Multiple LED's with optical emission at a wavelength of 405 nm can be used to construct a source that delivers about 2.5 Watts of optical power. A 2.5 Watt source delivers about 25 Joules of energy to a 1 cm² region of the skin in 10 seconds. This is approximately equivalent to the dose delivered by the aforementioned ClearLight device in a single 15-minute treatment. Available LED's are currently about 10% efficient at converting electrical light to optical power so that about 250 Joules of waste heat would be generated for a 25 Joule treatment dose.

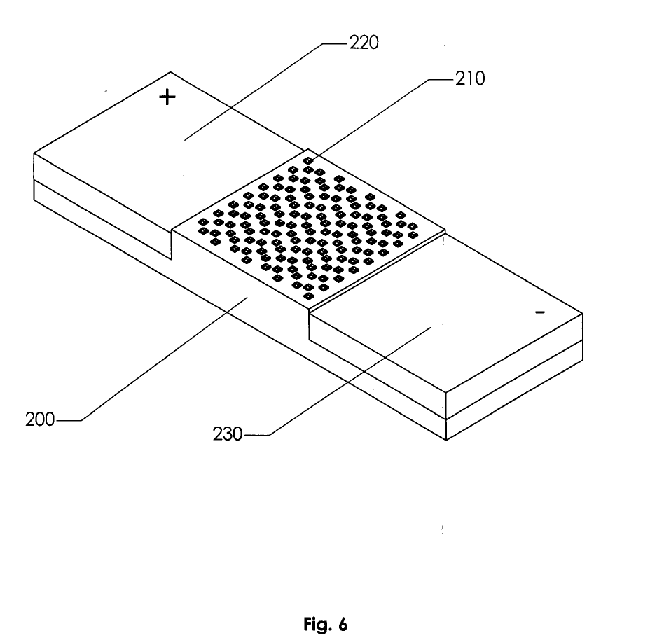

One embodiment of a two-dimensional LED light source is shown schematically in FIG. 6. In this embodiment, the light source is a two dimensional array of 128 light emitting diode dice 210, such as available from Medical Lighting Systems, Inc. (Tampa, Fla.). The dice are the raw semiconductor light-emitting device, by which it is meant that the die are not part of an assembly or package, and therefore do not include lenses. In this application, the foregoing are referred to as “unlensed” LED's. Note that commercial LED's are often sold as lamp assemblies that include the die, a substrate upon which the die is mounted, electrical leads, and an encapsulation that is shaped to form a lens. In this embodiment of the present invention, the dice are bonded to a copper heatsink 200 with thermally conductive epoxy that serves to remove heat from the die when they are energized. Electrical contact to the dice are made with wire-bonds, with 32 parallel strands each having four die connected in series. Each series is wire-bonded to a positively-charged busbar 220 and a negatively-charged busbar 230 such that current flows through the series of four dice. The busbars are electrically isolated from the copper heatsink. This configuration requires a supply voltage of approximately 16V. Each die has nominally 4.5 mW of optical output at 405 run with 20 mA of drive current, which provides about 575 mW of intense violet-blue light from the array. The dice may be driven with substantially higher current than 20 mA to yield a light source approaching 2.5 W, without an excessive reduction of lifetime, as long as adequate cooling is provided. Such adequate cooling may take the form of good coupling to the copper heatsink, and even thermally coupling the heat sink to another heat removal element. Note that LED's typically have very long lifetimes at the rated current, so that a reduction of lifetime may well be acceptable in practice.

In another embodiment, violet-blue diode lasers could be used as light source 20. For example, Nichia America, Inc. (Mountville, Pa.) manufactures diode lasers with 30 mW of optical output with peak wavelengths available in the 400-415 nm band with 70 mA of drive current (Nichia part no. NDHV310ACA). Therefore, a light source of 100 mW, 500 mW, and 2.5W of intense violet-blue light could be created by an array of about 3, 16, or 83 laser diodes, respectively. As with the LED's, the laser diodes could be driven with a higher current if well-coupled to an adequate heatsink and/or if a reduction of lifetime is acceptable, reducing the number of diode lasers required. In addition, violet-blue diode lasers are currently in an active area of research with regular performance improvements, making diode lasers an increasingly viable light source in the present invention.

The light source of the present invention most preferably has an output concentrated in the wavelength band of approximately 400-420 nm which generally matches the absorption peak of the porphyrins believed to be most prevalent in the acne regions. This band also generally matches the in vitro action spectrum reported by Kjeldstad and Johnsson (1986), which has a peak around 412-415 nm. However, the output could also be in a broader wavelength band from 400-450 nm.

The light source preferably has an output power of at least 100 mW/cm² in the violet-blue band, but more preferably has an output power of at least 500 mW/cm² in the violet-blue band.

In still another embodiment of the invention, alternate constructions of light source 20 could be used. Additional embodiments could also emit light energy in wavelength bands in addition to the violet-blue band, such as green or yellow bands that may also have porphyrin absorption or red bands that are believed to have anti-inflammatory benefits.

In the preferred embodiment shown in FIG. 1, mixer 30 also has the function of transferring heat absorbed by output window 10 to a thermal battery 40. The heat transfer of mixer 30 should be high enough to ensure that the heat conducted from the skin and deposited in window 10 during a previous exposure has been substantially removed from window 10 prior to the commencement of a subsequent exposure. In an alternate embodiment of the current invention, the functions of mixer 30, namely light mixing and heat transfer, could be performed by two distinct components.

A preferred embodiment of the device would also employ the use of a temperature sensor 50 to ensure that the assembly comprised of window 10, mixer 30, light source 20, and thermal battery 40 are not at an excessive temperature prior to the commencement of a treatment pulse. An excessive temperature may be reached after several treatment pulses. A temperature sensor is more important in an embodiment of the device that cools the window 10 below room temperature prior to illumination. In such an embodiment, it may be desirable to have temperature sensor 50 closer to window 10 to ensure the window is at the proper temperature prior to contact with the skin.

The preferred embodiment of the present invention also has a thermal battery 40 that is composed substantially of a material with sufficient heat capacity as to allow the device to work for tens or hundreds of ten-second pulses with a temperature rise of less than 10° C. This heat removal element may be simply be a mass of metal. Alternatively, a material that undergoes a phase change near room temperature can be used. These phase change materials can absorb large amounts of heat with little temperature increase. Optimized materials designed for phase change near room temperature or near skin temperature are available from several manufacturers, such as TEAP Energy (Perth, Australia). These materials could be contained within a metal housing designed to efficiently transfer the heat to the phase change material. Phase change materials with energy densities of about 50 J/cm³/° C. are readily available. A thermal battery that accepts the waste heat of over 100 exposures is inexpensive and is easily contained within a hand held device. Another type of thermal battery involves the use of a compressed substance, such as CO₂, which cools upon expansion and can thereby absorb heat energy from a higher temperature source.

A thermal battery 40 of the device may be “re-charged” by simply allowing the device to sit in a room-temperature environment, by placing the device into a refrigerator, or by placing the device in contact with a second device designed to actively conduct heat from thermal battery 40, by replacing or re-pressurizing the compressed substance, or by some other recharging mechanism.

Another embodiment of the current invention contains a finned heat sink and fan to more efficiently reject heat from the thermal battery into the room. A heat sink and fan that requires less than 1 Watt and fits into a hand-held device are available from several manufacturers, including Wakefield Thermal Solutions (Pelham, N.H.). Although the finned heatsink may be open to the air outside the housing, the element is to be considered inside the housing.

Still another embodiment of the current invention uses a thermoelectric cooler module, also known as a Peltier-effect device, such as available from Melcor (Trenton, N.J.) to remove heat from thermal battery 40. A device using a thermoelectric cooler module requires a small thermal battery or even no thermal battery at all.

Still another embodiment of the current invention contains a finned heat sink and fan as a heat removal element to reject heat directly from the device. For example, the light source and the output window may be thermally coupled directly to a finned heatsink that is air-cooled by a fan. Such an embodiment could operate in a steady-state condition where the device does not need to be thermally recharged and could operate indefinitely from a heat transfer standpoint. This embodiment could also use a thermoelectric cooler module.

The preferred embodiment of the invention also contains an electrical battery 60 and control electronics 70. Batteries with energy densities greater than 500 J/cm³ are readily available and a battery that powers the current invention for more than 100 exposures is inexpensive and is easily contained within a hand-held device. An alternative embodiment could be powered from mains power rather than from a battery or battery pack.

It is possible that the light output of some embodiments of the present invention may not be eye safe without mitigation, particularly in the case of diode laser-based light sources. In this event, preferred embodiments would employ an optical diffuser so that an integrated radiance of the light is reduced to an eye safe value. The diffuser may include a transmissive diffuser, such as PTFE or opal glass, and may include a reflective diffuser, such as Spectralon (Labsphere, Inc., North Sutton, N.H.). Other embodiments, such as an array of unlensed LED's similar to that described above, are expected to be inherently eye safe at the output power levels discussed herein, and would not require an optical diffuser.

A preferred embodiment of the present invention would also include a contact sensor that would enable light emission only when the device is in substantial contact with a surface, including the surface of the skin. Most preferably the contact sensor is indicative of contact between the output window 10 and the skin, thereby helping to ensure that the output window 10 provides an effective heatsink for the skin. A contact sensor may also act to reduce emission into the ambient environment that may be uncomfortably bright or may even not be eye safe. A contact sensor could be made of mechanical switches, capacitive switches, piezoelectric materials, or other approaches, and may include sensors located around the periphery of the output window 10. The contact sensor also preferably works only on compliant materials such as skin, so that contact with eyeglasses or flat transparent surfaces would not result in a positive indication of contact. This could be achieved, for example, by recessing the actuation buttons of a contact sensor below the emitting surface of window 20, such that contact with a flat, hard surface would not actuate the buttons. Also most preferably the contact sensor acts as a trigger for light emission, such that light emission would be automatically triggered when substantial contact is made with the skin. The light emission may be terminated after a fixed exposure time or if contact is broken or for other reasons. An automatic trigger upon contact is convenient for the user and removes the requirement for a separate trigger, such as one actuated by a finger.

A preferred embodiment of a battery-powered embodiment is one in which the battery would directly power the light source in a direct drive configuration. By “directly power” and “direct drive” it is intended to mean that the instantaneous current flowing through the battery and the instantaneous current flowing through the light source at a particular moment in time are substantially equivalent. The instantaneous currents differ only in that a comparatively small amount of current drawn from the battery is used to power the non-light-source components, such as the control electronics.

Further discussion and details about heat removal elements, thermal batteries, heatsinks, battery packs, optical diffusers, and direct drive battery powered configurations, and circuitry for controlling the above components, suitable for use in the present invention can be found in the above mentioned Cross-Referenced Non-Provisional Applications.

Detailed Thermal Calculations

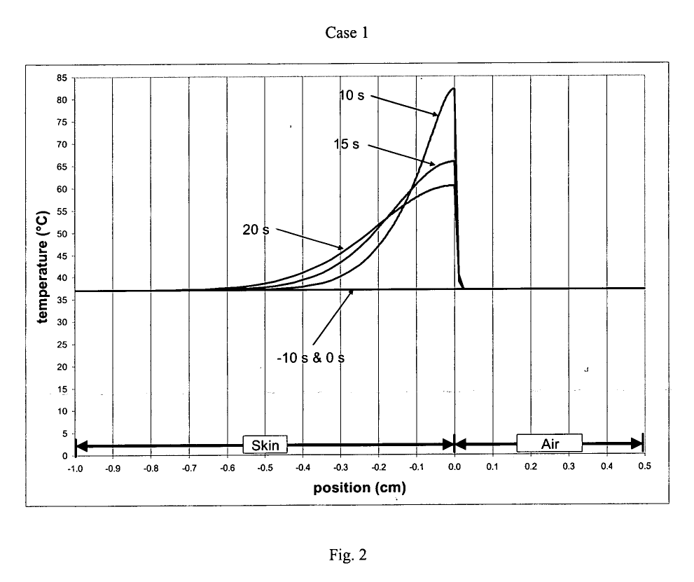

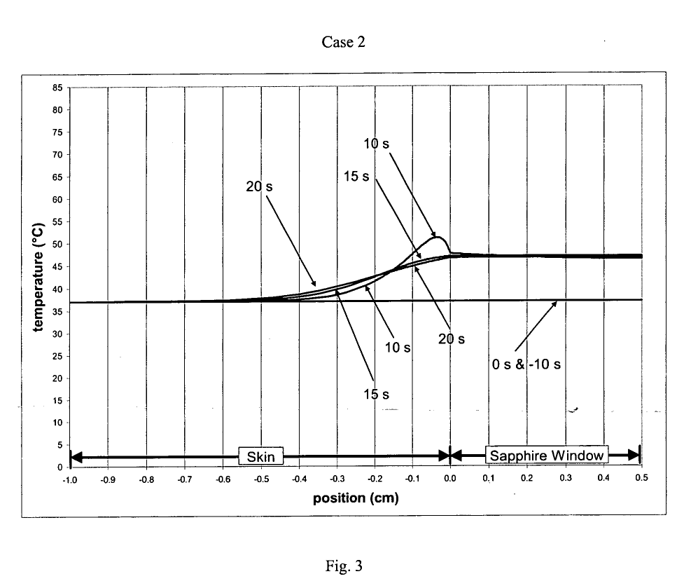

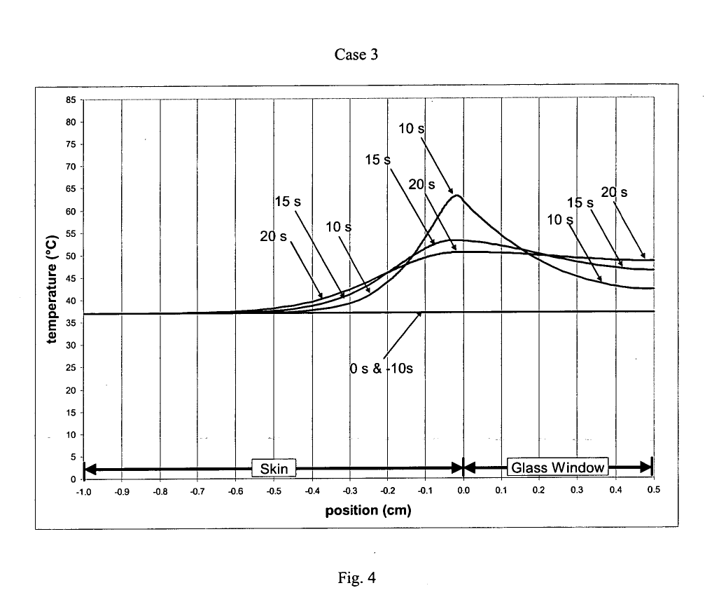

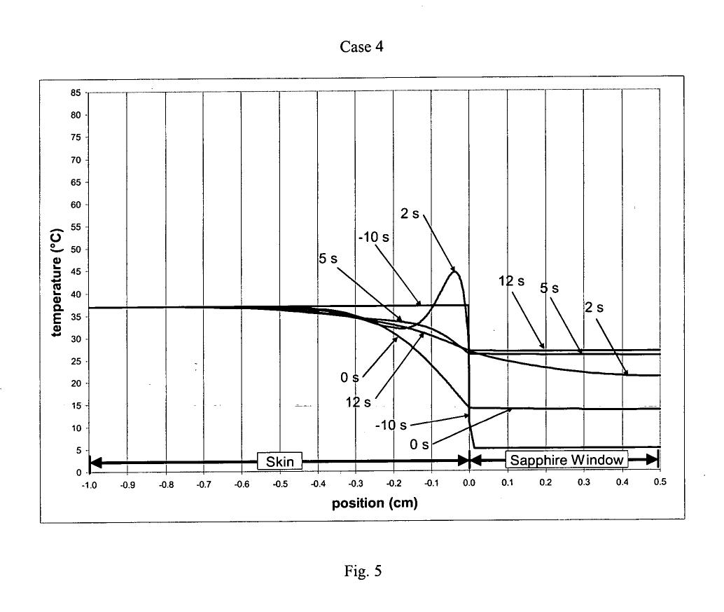

A finite element model of the device and of skin has been developed to simulate the heat transfer occurring prior to, during, and after light exposure of the skin. Many different cases have been modeled. Four cases have been included with this application. They are labeled Case 1, Case 2, Case 3, and Case 4 and the graphical results are shown in FIG. 2, FIG. 3, FIG. 4, and FIG. 5, respectively. The graphs contained in FIGS. 2-5 show the temperature of the skin and window versus position. Regions to the left of position x=0 are skin. Regions to the right of position x=0 are either air (Case 1) or the window contacting the skin (Case 2, Case 3, and Case 4).

In each case the initial temperature of the skin is 37° C. for the purposes of these calculations. In each case except for the first case, the output window of the device is touched to the skin at time t=−10 s and held in contact with the skin for 10 seconds prior to commencement of illumination of the skin. The first case simulates the treatment where the window is not held in contact with the skin so that there is only air in contact with the skin. In Case 2 and in Case 3, the initial temperature of the window is 37° C., representing the nominal skin temperature. In Case 4, the initial temperature of the window is 5° C. In each case, commencement of illumination occurs at time t=0 s. For cases 1, 2, and 3, the skin is illuminated with light for 10 s at an intensity of 2.5 W/cm². In the fourth case, the skin is illuminated for 2 s at an intensity of 12.5 W/cm². In each case an effective absorption length in skin of 0.3 mm was used to model the absorption of the incident light. This effective absorption length, 0.3 mm, is approximately that of 405 nm light in skin.

Notice from the graph of the results for Case 1 shown in FIG. 2 that when only air is in contact with the skin, the temperature of the skin reaches a maximum temperature of over 80° C. A temperature of 80° C. is above the threshold for damage to the skin and is painful.

The graph of the results for Case 2 in FIG. 3 shows that when a sapphire window with thickness of 5 mm and initial temperature of 37° C. is placed in contact with the skin for 10 s prior to the pulse of illumination, the maximum temperature of the skin is only approximately 52° C. This temperature is below the threshold for damage to the skin. It is perceived as hot but easily tolerated with little or no pain.

The graph of the results for Case 3 in FIG. 4 shows that a glass window with thickness of 5 mm and initial temperature of 37° C. does not perform as well as sapphire because of the limited thermal diffusivity of the glass. Notice the large temperature gradient in the glass window that existed at time, t=10 s, indicating that heat was not effectively transferred to the back surface of the glass during the illumination pulse. The maximum temperature of the skin in Case 3 is approximately 63° C.

Finally, the graph of the results for Case 4 in FIG. 5 shows that by cooling a sapphire window to 5° C. prior to contacting the skin, the maximum temperature of the skin is less than 45° C. even though the illumination of 12.5 W/cm² is much more intense than in the previous three cases.

From these simulations it is evident that a device with an output window placed in contact with the skin prior to or during the exposure of skin is effective at preventing thermal injury to the skin.

While exemplary drawings and specific embodiments of the present invention have been described and illustrated, it is to be understood that that the scope of the present invention is not to be limited to the particular embodiments discussed. Thus, the embodiments shall be regarded as illustrative rather than restrictive, and it should be understood that variations may be made in those embodiments by workers skilled in the arts without departing from the scope of the present invention, as set forth in the appended claims and structural and functional equivalents thereof.

In addition, in methods that may be performed according to preferred embodiments herein and that may have been described above, the operations have been described in selected typographical sequences. However, the sequences have been selected and so ordered for typographical convenience and are not intended to imply any particular order for performing the operations, unless expressly set forth in the claims or as understood by those skilled in the art as being necessary.

### Scientific Publications List

1. Kjeldstad B, et al., “Porphyrin photosensitization of bacteria,” Adv Exp Med Biol. 1985;193:155-9. PMID: 4096295 [PubMed—indexed for MEDLINE]

2. Arakane K, et al., “Singlet oxygen (1 delta g) generation from coproporphyrin in Propionibacterium acnes on irradiation,” Biochem Biophys Res Commun. 1996 Jun. 25;223(3):578-82. PMID: 8687438 [PubMed—indexed for MEDLINE]

3. Ashkenazi H, et al., “Eradication of Propionibacterium acnes by its endogenic porphyrins after illumination with high intensity blue light.” FEMS Immunol Med Microbiol. 2003 Jan. 21;35(1):17-24. PMID: 12589953 [PubMed—indexed for MEDLINE]

4. Cornelius CE 3rd, et al., “Red fluorescence of comedones: production of porphyrins by Corynebacterium acnes,” J Invest Dermatol. 1967 October;49(4):368-70. PMID: 4228644 [PubMed—indexed for MEDLINE]

5. Fanta D, et al., “Porphyrinsynthesis of Propionibacterium acnes in acne and seborrhea (author's transl),” Arch Dermatol Res. 1978 Apr. 7;261(2):175-9. German. PMID: 148872 [PubMed—indexed for MEDLINE]

6. Formanek I, et al., “[Porphyrinsynthesis by propionibacterium acnes (author's transl)],” Arch Dermatol Res. 1977 Aug. 22;259(2):169-76. German. PMID: 334087 [PubMed—indexed for MEDLINE]

7. Kawada A, et al., “Acne phototherapy with a high-intensity, enhanced, narrow-band, blue light source: an open study and in vitro investigation,” J Dermatol Sci. 2002 November;30(2):129-35. PMID: 12413768 [PubMed—indexed for MEDLINE]

8. Kjeldstad B, et al., “An action spectrum for blue and near ultraviolet inactivation of Propionibacterium acnes; with emphasis on a possible porphyrin photosensitization,” Photochem Photobiol. 1986 January;43(1):67-70. PMID: 3952162 [PubMed—indexed for MEDLINE]

9. Kjeldstad B, et al., “Influence of pH on porphyrin production in Propionibacterium acnes,” Arch Dermatol Res. 1984;276(6):396-400. PMID: 6517611 [PubMed—indexed for MEDLINE]

10. Lee W L, et al., “Comparative studies of porphyrin production in Propionibacterium acnes and Propionibacterium granulosum,” J. Bacteriol. 1978 February;133(2):811-5. PMID: 637914 [PubMed—indexed for MEDLINE]

11. McGinley K J, et al., “Facial follicular porphyrin fluorescence: correlation with age and density of Propionibacterium acnes,” Br J. Dermatol. 1980 April;102(4):437-41. PMID: 7387886 [PubMed—indexed for MEDLINE]

12. Meffert H, et al., “Therapy of acne with visible light. Decreased irradiation time by using a blue-light high-energy lamp [transl.],” Dermatol Monatsschr. 1990;176(10):597-603. German. PMID: 2150382 [PubMed—indexed for MEDLINE]

13. Meffert H, et al., “Phototherapy of acne vulgaris with the “TuR” UV 10 body section irradiation unit [transl.],” Dermatol Monatsschr. 1986;172(1):9-13. German. PMID: 2938991 [PubMed—indexed for MEDLINE]

14. Meffert H, et al., “Phototherapy of acne vulgaris with the UVA irradiation instrument TBG 400[transl.],” Dermatol Monatsschr. 1986;172(2):105-6. German. PMID: 2937663 [PubMed—indexed for MEDLINE]

15. Meffert H, et al., “Treatment of acne vulgaris with visible light [transl.],” Dermatol Monatsschr. 1987;173(11):678-9. German. PMID: 2963772 [PubMed—indexed for MEDLINE]

16. Kjeldstad B, et al., “Near-UV-induced radicals in Propionibacterium acnes, studied by electron spin resonance spectrometry at 77 K.,” J Photochem Photobiol B. 1991 May; 9(2):181-7. PMID: 1650821 [PubMed—indexed for MEDLINE]

17. Johnsson A, et al., “Fluorescence from pilosebaceous follicles,” Arch Dermatol Res. 1987;279(3):190-3. PMID: 3592747 [PubMed—indexed for MEDLINE]

18. Melo T B, et al., “Photodestruction of Propionibacterium acnes porphyrins,” Z Naturforsch [C]. 1985 Jan-Feb;40(1-2):125-8. PMID: 3993179 [PubMed—indexed for MEDLINE]

19. Melo T B, et al., “In vivo porphyrin fluorescence for Propionibacterium acnes. A characterization of the fluorescing pigments,” Dermatologica. 1982 March;164(3):167-74. PMID: 7084539 [PubMed—indexed for MEDLINE]

20. Mills O H, et al., “Ultraviolet phototherapy and photochemotherapy of acne vulgaris,” Arch Dermatol. 1978 February;114(2):221-3. PMID: 147054 [PubMed—indexed for MEDLINE]

21. Papageorgiou P, et al., “Phototherapy with blue (415 nm) and red (660 nm) light in the treatment of acne vulgaris,” Br J. Dermatol. 2000 May;142(5):973-8. PMID: 10809858 [PubMed—indexed for MEDLINE]

22. Romiti R, et al., “High-performance liquid chromatography analysis of porphyrins in Propionibacterium acnes,” Arch Dermatol Res. 2000 June;292(6):320-2. PMID: 10929774 [PubMed—indexed for MEDLINE]

23. Sigurdsson V, et al., “Phototherapy of acne vulgaris with visible light,” Dermatology. 1997;194(3):256-60. PMID: 9187844 [PubMed—indexed for MEDLINE]

24. Webster, G F, “Inflammation in acne vulgaris,” J Am Acad Dermatol. 1995 August;33(2 Pt 1):247-53. Review. PMID: 7622652 [PubMed—indexed for MEDLINE]

25. Fanta D, et al., “Porphyrin synthesis by propionibacteria in dependence of external factors.” Arch Dermatol Res (1981) 271:127-133

26. Leyden J, “Therapy for acne vulgaris,” New England Journal of Medicine, Apr. 17, 1997, Review Article

27. Shalita A, et al., “Acne photoclearing (APC) using a novel, high-intensity, enhanced, narrow-band, blue light source,” Clinical application notes Volume 9 Number 1, ESC Medical Systems Ltd (Yokneam, Israel) PB 558-0230 Rev. A

28. Shnitkind E, et al., “Anti-inflammatory properties of narrow band blue light,” Poster presentation (conference unknown)

29. Leung, S, “The Porphyrin Page” website at http://www.washburn.edu/cas/chemistry/sleung/porphyrin/porphyrin_page.html, Created Apr. 16, 1996, Last Modified Nov. 11, 2002.

We claim:

1. A dermatologic device for the treatment of acne, comprising
a light source producing light primarily in a wavelength band of 400 to 450 nm, and
an output window through which the light is emitted and which is configured to contact an epidermis of a subject undergoing treatment when the light is emitted through the output window, and
wherein the light has an intensity of at least 100 mW/cm² at an emitting surface of the output window, and
further wherein heat conduction through the output window is such that a temperature rise produced in the epidermis by the light is substantially reduced by the contact between the epidermis and the output window.

2. The dermatologic device of claim 1, wherein the light source comprises a diode light source.

3. The dermatologic device of claim 2, wherein the light source comprises one or more light emitting diodes.

4. The dermatologic device of claim 3, wherein the one or more light emitting diodes are unlensed, and are arranged in a two-dimensional array when two or more in number.

5. The dermatologic device of claim 2, wherein the light source comprises a diode laser.

6. The dermatologic device of claim 5, wherein the light propagates along a light path, and further including an optical diffuser positioned along the light path so that an integrated radiance of the light is reduced to an eye-safe value.

7. The dermatologic device of claim 6, wherein the optical diffuser is transmissive.

8. The dermatologic device of claim 6, wherein the optical diffuser is reflective.

9. The dermatologic device of claim 2, wherein the light has an intensity greater than 500 mW/cm² at the emitting surface.

10. The dermatologic device of claim 2, wherein the light has a spot size of less than 5 cm² at the emitting surface.

11. The dermatologic device of claim 2, wherein the output window is maintained at a temperature less than a nominal epidermis temperature.

12. The dermatologic device of claim 11, wherein the output window comprises a material selected from sapphire, fused quartz, fused silica, polymeric materials, or glass.

13. The dermatologic device of claim 12, wherein the output window comprises sapphire.

14. The dermatologic device of claim 2, wherein the output window is maintained at about a nominal epidermis temperature.

15. The dermatologic device of claim 14, wherein the output window comprises a material selected from sapphire, fused quartz, fused silica, polymeric materials, or glass.

16. The dermatologic device of claim 15, wherein the output window comprises sapphire.

17. The dermatologic device of claim 2, further including a contact sensor coupled to control the production of light by the light source so that light is emitted through the output window only when substantial contact is made between the contact sensor and a contacted surface.

18. The dermatologic device of claim 17, wherein the light source is automatically triggered and the light is emitted through the output window when the substantial contact is made.

19. The dermatologic device of claim 2, further including a thermal battery coupled to the output window, wherein the thermal battery comprises one or more of a metal, a phase change material, or a compressed substance.

20. The dermatologic device of claim 2, further including a thermoelectric cooler module.

21. A self-contained cordless dermatologic device for the treatment of acne, comprising
a housing configured for handheld manipulation;
one or more electrical batteries within the housing;
a light source within the housing and powered by the electrical batteries and producing light substantially in a wavelength band of 400 to 450 nm;
an output window through which the light is emitted and which is configured to contact an epidermis of a subject undergoing treatment when the light is emitted through the output window;
a heat-removal element within the housing;
wherein the light has an intensity of at least 100 mW/cm² at an emitting surface of the output window;
the cordless device has a total weight of less than 1 kilogram and has a volume less than 1500 cm³; and
a temperature rise produced in the epidermis by the light is substantially reduced by the contact between the epidermis and the output window.

22. The self-contained cordless dermatologic device of claim 21, wherein the heat removal element includes one or more thermal batteries, or one or more finned heat sinks, or both.

23. The self-contained cordless dermatologic device of claim 22, wherein the light source comprises a diode light source.

24. The self-contained cordless dermatologic device of claim 23, wherein the light source comprises one or more light emitting diodes.

25. The self-contained cordless dermatologic device of claim 24, wherein the one or more light emitting diodes are unlensed, and are arranged in a two-dimensional array when two or more in number.

26. The self-contained cordless dermatologic device of claim 23, wherein the light source comprises a diode laser.

27. The self-contained cordless dermatologic device of claim 26, wherein the light propagates along a light path, and further including an optical diffuser positioned along the light path so that an integrated radiance of the light is reduced to an eye-safe value.

28. The self-contained cordless dermatologic device of claim 27, wherein the optical diffuser is transmissive.

29. The self-contained cordless dermatologic device of claim 27, wherein the optical diffuser is reflective.

30. The self-contained cordless dermatologic device of claim 23, wherein the light has an intensity greater than 500 mW/cm² at the emitting surface.

31. The self-contained cordless dermatologic device of claim 23, wherein the light has a spot size of less than 5 cm² at the emitting surface.

32. The self-contained cordless dermatologic device of claim 23, wherein the output window is maintained at a temperature less than a nominal epidermis temperature.

33. The self-contained cordless dermatologic device of claim 32, wherein the output window comprises a material selected from sapphire, fused quartz, fused silica, polymeric materials, or glass.

34. The self-contained cordless dermatologic device of claim 33, wherein the output window comprises sapphire.

35. The self-contained cordless dermatologic device of claim 23, wherein the output window is maintained at about a nominal epidermis temperature.

36. The self-contained cordless dermatologic device of claim 35, wherein the output window comprises a material selected from sapphire, fused quartz, fused silica, polymeric materials, or glass.

37. The self-contained cordless dermatologic device of claim 36, wherein the output window comprises sapphire.

38. The self-contained cordless dermatologic device of claim 23, further including a contact sensor coupled to control the production of light by the light source so that light is emitted through the output window only when substantial contact is made between the contact sensor and a contacted surface.

39. The self-contained cordless dermatologic device of claim 38, wherein the light source is automatically triggered and the light is emitted through the output window when the substantial contact is made.

40. The self-contained cordless dermatologic device of claim 23, further including a thermal battery coupled to the output window, wherein the thermal battery comprises one or more of a metal, a phase change material, or a compressed substance.

41. The self-contained cordless dermatologic device of claim 23, further including a thermoelectric cooling module.

42. The self-contained cordless dermatologic device of claim 23, further including a direct drive electrical circuit for energizing the light source.

43. A dermatologic device for treatment of acne, comprising
a diode light source producing light in a wavelength band of 400 to 450 nm;
an output window through which the light is emitted and which is configured to contact an epidermis of a subject undergoing treatment when the light is emitted through the output window, and
wherein the light has an intensity of at least 100 mW/cm² at an emitting surface of the output window;
the light at the emitting surface has a spot size of less than 25 cm²; and
a temperature rise produced in the epidermis by the light is substantially reduced by the contact between the epidermis and the output window.

44. The dermatologic device of claim 43, wherein the diode light source comprises one or more light emitting diodes.

45. The dermatologic device of claim 44, wherein the one or more light emitting diodes are unlensed, and are arranged in a two-dimensional array when two or more in number.

46. The dermatologic device of claim 43, wherein the light source comprises a diode laser.

47. The dermatologic device of claim 46, wherein the light propagates along a light path, and further including an optical diffuser positioned along the light path so that an integrated radiance of the light is reduced to an eye-safe value.

48. The dermatologic device of claim 47, wherein the optical diffuser is transmissive.

49. The dermatologic device of claim 47, wherein the optical diffuser is reflective.

50. The dermatologic device of claim 43, wherein the light has an intensity greater than 500 mW/cm² at the emitting surface.

51. The dermatologic device of claim 43, wherein the light has a spot size of less than 5 cm² at the emitting surface.

52. The dermatologic device of claim 43, wherein the output window is maintained at a temperature less than a nominal epidermis temperature.

53. The dermatologic device of claim 52, wherein the output window comprises a material selected from sapphire, fused quartz, fused silica, polymeric materials, or glass.

54. The dermatologic device of claim 53, wherein the output window comprises sapphire.

55. The dermatologic device of claim 43, wherein the output window is maintained at about a nominal epidermis temperature.

56. The dermatologic device of claim 55, wherein the output window comprises a material selected from sapphire, fused quartz, fused silica, polymeric materials, or glass.

57. The dermatologic device of claim 56, wherein the output window comprises sapphire.

58. The dermatologic device of claim 43, further including a contact sensor coupled to control the production of light by the light source so that light is emitted through the output window only when substantial contact is made between the contact sensor and a contacted surface.

59. The dermatologic device of claim 58, wherein the light source is automatically triggered and the light is emitted through the output window when the substantial contact is made.

60. The dermatologic device of claim 43, further including a thermal battery coupled to the output window, wherein the thermal battery comprises one or more of a metal, a phase change material, or a compressed substance.

61. The dermatologic device of claim 43, further including a thermoelectric cooling module.

62. A self-contained cordless device for treating acne, comprising
a housing configured for handheld manipulation;
a diode light source within the housing producing light in a wavelength band of 400 to 450 nm;
one or more electrical batteries within the housing coupled to power the diode light source;
a heat removal element within the housing comprising one or more thermal batteries within the housing, or one or more finned heat sinks within the housing, or both;
an output window through which the light is emitted and which is configured to contact an epidermis when the light is emitted through the output window;
wherein the light has an intensity of at least 100 mW/cm² at an emitting surface of the output window;
the light has a spot size of less than 10 cm² at the emitting surface of the output window; and
a temperature rise produced in the epidermis by the light is substantially reduced by the contact between the epidermis and the output window.

63. The self-contained cordless dermatologic device of claim 62, wherein the diode light source comprises one or more light emitting diodes.

64. The self-contained cordless dermatologic device of claim 63, wherein the one or more light emitting diodes are unlensed, and are arranged in a two-dimensional array when two or more in number.

65. The self-contained cordless dermatologic device of claim 62, wherein the light source comprises a diode laser.

66. The self-contained cordless dermatologic device of claim 65, wherein the light propagates along a light path, and further including an optical diffuser positioned along the light path so that an integrated radiance of the light is reduced to an eye-safe value.

67. The self-contained cordless dermatologic device of claim 66, wherein the optical diffuser is transmissive.

68. The self-contained cordless dermatologic device of claim 66, wherein the optical diffuser is reflective.

69. The self-contained cordless dermatologic device of claim 62, wherein the light has an intensity greater than 500 mW/cm² at the emitting surface.

70. The self-contained cordless dermatologic device of claim 62, wherein the light has a spot size of less than 5 cm² at the emitting surface.

71. The self-contained cordless dermatologic device of claim 62, wherein the output window is maintained at a temperature less than a nominal epidermis temperature.

72. The self-contained cordless dermatologic device of claim 71, wherein the output window comprises a material selected from sapphire, fused quartz, fused silica, polymeric materials, or glass.

73. The self-contained cordless dermatologic device of claim 72, wherein the output window comprises sapphire.

74. The self-contained cordless dermatologic device of claim 62, wherein the output window is maintained at about a nominal epidermis temperature.

75. The self-contained cordless dermatologic device of claim 74, wherein the output window comprises a material selected from sapphire, fused quartz, fused silica, polymeric materials, or glass.

76. The self-contained cordless dermatologic device of claim 75, wherein the output window comprises sapphire.

77. The self-contained cordless dermatologic device of claim 62, further including a contact sensor coupled to control the production of light by the light source so that light is emitted through the output window only when substantial contact is made between the contact sensor and a contacted surface.

78. The self-contained cordless dermatologic device of claim 77, wherein the light source is automatically triggered and the light is emitted through the output window when the substantial contact is made.

79. The self-contained cordless dermatologic device of claim 62, wherein the thermal battery comprises one or more of a metal, a phase change material, or a compressed substance.

80. The self-contained cordless dermatologic device of claim 62, further including a thermoelectric cooling module.

81. The self-contained cordless dermatologic device of claim 62, further including a direct drive electrical circuit for energizing the light source.

82. The dermatologic device of claim 1, wherein the temperature rise produced in the epidermis is no more than 25° C.

83. The self-contained cordless dermatologic device of claim 21, wherein the temperature rise produced in the epidermis is no more than 25° C.

84. The dermatologic device of claim 43, wherein the temperature rise produced in the epidermis is no more than 25° C.

85. The self-contained cordless device of claim 62, wherein the temperature rise produced in the epidermis is no more than 25° C.

86. A method for the treatment of acne, comprising
positioning an output window to make contact with an epidermis of a subject undergoing treatment;
emitting light in a wavelength band of 400 to 450 nm through the output window, wherein the light has an intensity of at least 100 mW/cm² at an emitting surface of the output window, and
conducting heat through the output window so that a temperature rise produced in the epidermis by the light is substantially reduced by the contact between the epidermis and the output window.

87. The method of claim 86, including the step of maintaining the output window at a temperature less than a nominal epidermis temperature.

88. The method of claim 86, including the step of maintaining the output window at about a nominal epidermis temperature.

89. The method of claim 86, wherein the conducting heat step comprises conducting sufficient heat so that the temperature rise produced in the epidermis is no more than 25° C.

90. The method of claim 86, further including the step of inhibiting emission of light through the output window unless contact between the output window and the epidermis is sensed by a contact sensor.

91. The method of claim 86, further including the step of diffusing the light so that an integrated radiance of the light is reduced to an eye-safe level.

92. A method for configuring a device for treating acne, comprising
(a) providing within a housing configured for handheld manipulation
(i) a diode light source producing light in a wavelength band of 400 to 450 nm;
(ii) one or more electrical batteries;
(iii) a heat removal element; and
(iv) an output window through which the light is emitted;
(b) powering the diode light source solely from the one or more electrical batteries so that the light has an intensity of at least 100 mW/cm² at an emitting surface of the output window;
(c) configuring the output window for contact with an epidermis; and
(d) providing a heat conduction through the output window so that a temperature rise produced in the epidermis by the light is substantially reduced by contact between the epidermis and the output window.

93. The method of claim 92, wherein the providing heat conduction step comprises conducting sufficient heat so that the temperature rise produced in the epidermis is no more than 25° C.

94. The method of claim 92, including the step of maintaining the output window at a temperature less than a nominal epidermis temperature.

95. The method of claim 92, including the step of maintaining the output window at about a nominal epidermis temperature.

96. The method of claim 92, further including the step of positioning an output sensor on the housing so that emission of light through the output window is inhibited unless contact between the output window and the epidermis is sensed by the contact sensor.

97. The method of claim 92, further including the step of configuring the light emitted at the emitting surface of the output window to have a spot size of less than 10 cm².

98. The method of claim 92, further including the step of diffusing the light so that an integrated radiance of the light is reduced to an eye-safe level.

99. A method for operating a device for treating acne, wherein the device includes a housing configured for handheld manipulation, a diode light source producing light in a wavelength band of 400 to 450 nm, one or more electrical batteries, a heat removal element; and an output window through which the light is emitted, wherein the output window is configured for contact with an epidermis, the method comprising the steps of
(a) powering the diode light source solely from the one or more electrical batteries;
(b) energizing the diode light source so that light is produced with an intensity of at least 100 mW/cm² at an emitting surface of the output window;
(c) providing a heat conduction through the output window so that a temperature rise produced in the epidermis by the light is substantially reduced by contact between the epidermis and the output window.

100. The method of claim 99, further including the step of inhibiting emission of light through the output window unless contact between the output window and the epidermis is sensed by a contact sensor.

101. The method of claim 99, further including the step of diffusing the light so that an integrated radiance of the light is reduced to an eye-safe level.

102. The method of claim 99, wherein the providing heat conduction step comprising thermally coupling the heat removal element to the output window.

103. The method of claim 99, further including the step of configuring the light emitted at the emitting surface of the output window to have a spot size of less than 10 cm².
